(19) United States
(12) Patent Application Publication
(10) Pub. No.: US 2004/0161436 A1
(43) Pub. Date: Aug. 19, 2004

(54) Skin treatment composition and method

(76) Inventor: Barbara A. Hull, Irving, TX (US)

Correspondence Address: GRADY K. BERGEN, 2626 COLE AVENUE, SUITE 400, DALLAS, TX, 75204, US

(21) Appl. No.: 10/366,280

(22) Filed: Feb. 13, 2003

(51) Int. Cl. A61K 31/355; A61K 31/01; A61K 7/00

(52) U.S. Cl. 424/401; 514/458; 514/762

(57) ABSTRACT

A skin treatment composition and method is provided for improving the appearance of the skin. The composition includes d-limonene in a dermatological suitable vehicle that can be applied to the skin. The d-limonene may be present in an amount of 15% or less by weight of the composition.

### TECHNICAL FIELD

The invention relates generally to skin treatment.

### DETAILED DESCRIPTION

D-limonene is a naturally occurring substance that may be found in many citrus fruits, such as oranges, lemons, grapefruits, etc., as well as other sources, and may be derived or extracted from the pulp, peels or rinds of such fruits. D-limonene has been used as a solvent/degreaser and in cleaning products and soaps. Additionally, d-limonene has been used in insecticides and insect repellants. It is particularly attractive for such uses because of its low toxicity to humans and low environmental impact. It also has use as a flavoring and as a fragrance additive.

It has been found that d-limonene has particular application in treating the skin of humans to improve its appearance. When applied to the skin in a suitable manner, as is described herein, it has been observed to reduce or eliminate certain discolorations, such as redness, rosacea and bruises. It also facilitates the reduction of skin aging effects. In particular, application of d-limonene may result in the tightening of the skin, smoothing of lines and wrinkles, closing or contraction of skin pores, and smoothing of cellulite. It may also be used to reduce or eliminate acne and pimples, as well as other skin disorders such as eczema. The composition may also provide a fresh feeling and tingling sensation to the user's skin. It also provides a pleasant odor.

In order to achieve the above-described effects, the d-limonene is used in a dermatological suitable vehicle that can be applied to the human skin and is non-toxic. Such vehicles are well known by those in the cosmetic and pharmaceutical industries and may be aqueous or non-aqueous. The vehicles may be oil or water or a mixture thereof. The vehicle may provide a low viscosity composition, such that the composition is readily pourable or even sprayable, or may be of higher viscosity or even semisolid, such as an ointment or gel. Suitable vehicles may include, but are not limited to water, alcohol, (e.g. ethanol, isopropanol, etc.), organic solvents, hydrocarbon-based materials, such as petrolatum, etc. The vehicle may be used to form a suspension or solution.

When combined with the vehicle and any other additives, the composition may be in the form of a pourable or sprayable liquid, a lotion, ointment or a gel that may be applied to the skin. The d-limonene may be used in an amount of about 15% or less by weight of the composition, more typically about 10% or less or 5% or less by weight of the composition. At higher concentrations, the d-limonene may irritate the skin to a degree that makes its use undesirable at such levels. A suitable range for the d-limonene is from about 1% to about 15% by weight of the composition, with from about 2%, 3%, 4% or 5% to about 10% by weight of the composition being more typical.

The composition may optionally contain other compounds as well. This may include vitamin compounds, such as vitamin E or tocopherol, which may be in the form most commonly used in topical applications, such as alpha-tocopherol acetate and alpha-tocopherol succinate. The vitamin E may be used in an amount of from about 1% or 0.5% or less by weight of the composition.

The composition may also contain other ingredients such as emulsifiers, stabilizers, preservatives, colorants, fragrance additives, etc.

Tea tree oil and/or emu oil may be added to the composition in amounts of from about 0.1 to about 5% by weight of the composition. Such composition may be used to facilitate and/or enhance shaving or smoothing of the skin.

The composition may be applied to the skin in any suitable manner. The composition may be delivered from its container by spraying, dousing, pouring or squeezing the composition from the container. If sprayed, it may be desirable to deliver the composition in a manner that minimizes or eliminates droplets or particles from such spray that might otherwise irritate the eyes or be inhaled. The composition may be applied with an applicator, such as a sponge, cloth, cotton ball or other article soaked or bearing the composition or be applied to the hands and rubbed onto the area to be treated. If necessary, the composition may be further spread onto and/or rubbed into the skin so that the area of treatment is effectively covered.

The composition may be applied one or more times a day. If applied more than once a day, the composition may be applied at different intervals, such as approximately every 2, 3, 4, 6, 8 or 12 hours. The frequency of application needed may be determined by observing the effectiveness of the application. If no visible improvements in the skin are seen, the user may increase the frequency of application. Likewise, the frequency of application may be reduced if the treated area shows improvement soon after application.

The following examples serve to further illustrate the invention.

### EXAMPLES

A skin treatment composition was prepared by combining a concentrated d-limonene solution, available as CITRIX, from Envirosafe Labs, LLC, Conroe, Tex., which was a 79.2% d-limonene solution with distilled water in a ratio of approximately 1:10 by weight, respectively. This composition was then used as set forth in the following examples.

### Example 1

The above solution was applied from approximately 1 to 4 times a day to the skin of a 71 year old male human subject over a period of approximately 1½ months. After such period, the subject observed fewer wrinkles, smoother skin and smaller skin pores, particularly those around the nose and eyes. The subject also observed healthy glow to the skin and fading of brown spots.

### Example 2

The above composition was applied from approximately 1 to 4 times a day to the skin of a 55 year old female human subject. The solution was applied to the skin of the face with a cotton ball and applied to the rest of the subject's body by spraying. After application of the solution for a period of time, the subject observed significant reduction in pore size on the skin of the cheeks and chin. Reduction in wrinkles was also observed, particularly around the neck of the subject.

### Example 3

A 24 year old female subject applied the composition to the face of her skin from approximately 1 to 4 times a day. After the second day of application, the subject observed significant smoothing of acne scars and bumps. After the application of the composition was discontinued, the subject observed a return of the acne scars and bumps.

### Example 4

The composition was applied twice a day to various areas of the skin of a female subject. Prior to application, the subject observed six areas exhibiting dark growths of skin nevus. After application of the composition of approximately two months, the subject observed only one area remaining area of skin nevus remaining. The subject also observed elimination of skin rosacea and lightening of brown spots on their hands, arms and face. The subject also applied the composition twice a day to areas of the feet and toes exhibiting eczema with elimination and control of the eczema symptoms, including itching. The eczema was controlled by commencing application of the composition upon the recurrence of itching caused by the eczema.

### Example 5

A 36 year old male subject applied the composition from 1 to 4 times a day to areas of the skin exhibiting severe rosacea that had been present for several years. Significant improvements in the skin's appearance were observed soon after application. The subject also used the same composition additionally containing tea tree oil and emu oil and observed that the composition facilitated and enhanced shaving.

While the invention has been shown in only some of its forms, it should be apparent to those skilled in the art that it is not so limited, but is susceptible to various changes and modifications without departing from the scope of the invention. Accordingly, it is appropriate that the appended claims be construed broadly and in a manner consistent with the scope of the invention.

I claim:

1. A topical composition for treatment of the skin to improve the skin's appearance comprising:
d-limonene in a dermatological suitable vehicle that can be applied to the skin.

2. The composition of claim 1, wherein:
the vehicle is selected from a group consisting of water, alcohol, organic solvents andhydrocarbon-based materials.

3. The composition of claim 1, wherein:
the d-limonene is present in an amount of about 15% or less by weight of the composition.

4. The composition of claim 1, wherein:
the d-limonene is present in an amount of about 10% or less by weight of the composition.

5. The composition of claim 1, further comprising;
tocopherol (vitamin e).

6. The composition of claim 5, wherein:
the tocopherol is present in an amount of 1% or less by weight of the composition.

7. The composition of claim 5, wherein:
the tocopherol is present in an amount of 0.5% or less by weight of the composition.

8. A topical composition for applying to the skin for treatment of at least one of acne, aging effects of the skin, smoothing of lines or wrinkles, rosacea, bruises, open pores, redness and smoothing of cellulite, the composition comprising:
d-limonene in a dermatological suitable vehicle that can be applied to the skin, the d-limonene being present in amount of about 15% or less by weight of the composition.

9. The composition of claim 8, wherein:
the vehicle is selected from a group consisting of water, alcohol, organic solvents and hydrocarbon-based materials.

10. The composition of claim 8, wherein:
the d-limonene is present in an amount of about 10% or less by weight of the composition.

11. The composition of claim 8, wherein:
the d-limonene is present in an amount of from about 10% to about 1% by weight of the composition.

12. The composition of claim 8, further comprising;
tocopherol (vitamin e).

13. The composition of claim 12, wherein:
the tocopherol is present in an amount of 1% or less by weight of the composition.

14. The composition of claim 12, wherein:
the tocopherol is present in an amount of 0.5% or less by weight of the composition.

15. A method of treating the skin comprising to improve the skin's appearance comprising:
providing a composition containing d-limonene in a dermatological suitable vehicle; and
applying the composition to the skin.

16. The method of claim 15, wherein:
applying the composition to the skin includes applying to an area of the skin exhibiting at least one of acne, aging effects, lines, wrinkles, rosacea, bruises, redness and cellulite.

17. The method of claim 15, wherein:
the d-limonene is present in the composition in an amount of about 15% or less by weight of the composition.

18. The method of claim 15, wherein:
the composition further includes tocopherol.

19. The method of claim 15, wherein:
the dermatological suitable vehicle includes water, alcohol, organic solvents and hydrocarbon-based materials.
